SEQUENCE LISTING (3 sequences)

SEQ ID NO: 1 - protein, 500 aa, Thermus thermophilus
MELPRAFGLLLHPTSLPGPYGVGVLGQEARDFLRFLKEAGGRYWQVLPLGPTGYGDSPYQSFSAFAGNPYLIDLRPLAERGYVRLEDPGFPQGRVDYGLLYAWKWPALKEAFRGFKEKASPEEREAFAAFREREAWWLEDYALFMALKGAHGGLPWNRWPLPLRKREEKALREAKSALAEEVAFHAFTQWLFFRQWGALKAEAEALGIRIIGDMPIFVAEDSAEVWAHPEWFHLDEEGRPTVVAGVPPDYFSETGQRWGNPLYRWDVLEREGFSFWIRRLEKALELFHLVRIDHFRGFEAYWEIPASCPTAVEGRWVKAPGEKLFQKIQEVFGEVPVLAEDLGVITPEVEALRDRFGLPGMKVLQFAFDDGMENPFLPHNYPAHGRVVVYTGTHDNDTTLGWYRTATPHEKAFMARYLADWGITFREEEEVPWALMHLGMKSVARLAVYPVQDVLALGSEARMNYPGRPSGNWAWRLLPGELSPEHGARLRAMAEATERL

SEQ ID NO: 2 - protein, 238 aa, artificial sequence
  Synthetic Construct
MSKGEELFTGVVPILVELDGDVNGQKFSVSGEGEGDATYGKLTLKFICTTGKLPVPWPTLVTTLTYGVQCFSRYPDHMKQHDFFKSAMPEGYVQERTIFYKDDGNYKTRAEVKFEGDTLVNRIELKGIDFKEDGNILGHKMEYNYNSHNVYIMADKPKNGIKVNFKIRHNIKDGSVQLADHYQQNTPIGDGPVLLPDNHYLSTQSALSKDPNEKRDHMILLEFVTAAGITHGMDELYK

SEQ ID NO: 3 - protein, 326 aa, Pyrococcus furiosus
MKILLIGMGGTIASVKGENGYEASLSVKEVLDIAGIKDCEDCDFLDLKNVDSTLIQPEDWVDLAETLYKNVKKYDGIIVTHGTDTLAYTSSMISFMLRNPPIPIVFTGSMIPATEENSDAPLNLQTAIKFATSGIRGVYVAFNGKVMLGVRTSKVRTMSRDAFESINYPIIAELRGEDLVVNFIPKFNNGEVTLDLRHDPKVLVIKLIPGLSGDIFRAAVELGYRGIVIEGYGAGGIPYRGSDLLQTIEELSKEIPIVMTTQAMYDGVDLTRYKVGRLALRAGVIPAGDMTKEATVTKLMWILGHTNNVEEIKVLMRKNLVGELRD

(19) United States
(12) Patent Application Publication
(10) Pub. No.: US 2016/0122772 A1
(43) Pub. Date: May 5, 2016

(54) Production of Polypeptides Without Secretion Signal in Bacillus

(71) Applicant: Novozymes A/S, Bagsvaerd (DK)

(72) Inventors: Niels Banke, Bagsvaerd (DK); Michael Dolberg Rasmussen, Bagsvaerd (DK); Poul Erik Pedersen, Bagsvaerd (DK); Martin Simon Borchert, Bagsvaerd (DK)

(73) Assignee: Novozymes A/S, Bagsvaerd (DK)

(21) Appl. No.: 14/896,423

(22) PCT Filed: Jun. 23, 2014
(86) PCT No.: PCT/EP2014/063184

§ 371 Date: Dec. 7, 2015

Related U.S. Application Data
(60) Provisional application No. 61/837,995, filed on Jun. 21, 2013.

(51) Int. Cl. C12N 15/75; C12N 9/10; C12N 9/82; C12P 21/00

(52) U.S. Cl. CPC C12N 15/75; C12P 21/00; C12N 9/1051; C12N 9/82; C12Y 204/01; C12Y 305/01001

(57) ABSTRACT

The present invention relates to a method of producing a natively non-secreted polypeptide without a secretion signal in a Bacillus host cell and recovering the polypeptide without performing a lysis step as well as to a Bacillus host cell comprising one or more exogenous or heterologous polynucleotides encoding a natively non-secreted polypeptide with no secretion signal.

### FIELD OF THE INVENTION

The present invention relates to a method of producing a natively non-secreted polypeptide without a secretion signal in a Bacillus host cell and recovering the natively non-secreted polypeptide without performing a lysis step as well as to a Bacillus host cell comprising one or more exogenous or heterologous polynucleotide encoding a natively non-secreted polypeptide of interest with no secretion signal.

### BACKGROUND OF THE INVENTION

Bacillus host cells have been characterized as workhorses in the industrial manufacture of various polypeptides of interest, mainly because they actively secrete polypeptides that have a so-called signal peptide. The signal peptide is a small polypeptide, typically around 20-30 amino acids, that is expressed in transcriptional and translational fusion with the N-terminal of a polypeptide to be secreted. It directs the fused polypeptide into the secretory machinery of a suitably equipped host cell, whereupon the fused polypeptide is cleaved while the now-matured polypeptide of interest is secreted into the surrounding culture broth without its signal peptide which is retained in the cell and degraded.

The secretion of recombinantly produced polypeptides of interest in Bacillus enables a comparatively easy recovery of the polypeptides directly from the culture broth without having to perform a cell lysis step. Only polypeptides destined to be exported from the cell into the growth medium are natively outfitted with a signal peptide in Bacillus.

However, most polypeptides in a cell are not destined for export but are instead intended to be intracellular, periplasmic, membrane-bound etc. Such natively non-secreted polypeptides are usually considered burdensome to produce because they either need to be recovered from within the host cells which usually requires a messy cell-lysis step resulting in a challenging recovery process, or they need to be expressed as artificial fusion polypeptides together with a heterologous secretion signal in the hope that this will enable active secretion from a host cell like Bacillus which is by no means guaranteed to succeed. Accordingly, there is a need in the art for methods to produce natively non-secreted polypeptides in an economically efficient way.

### SUMMARY OF THE INVENTION

Contrary to our expectations, we found very high levels of enzyme activity in the supernatants when natively intracellular or non-secreted enzymes were expressed in Bacillus licheniformis and Bacillus subtilis hosts without a signal peptide This is a highly surprising result and one of economical importance, since this demonstrates that natively non-secreted polypeptides can be produced in Bacillus and be recovered directly from the broth without a costly cell lysis step.

A Bacillus subtilis strain over-expressing a natively non-secreted asparaginase (ASP) with no signal peptide and a Bacillus licheniformis strain over-expressing natively non-secreted glucanotransferase (GT) with no signal peptide or a GFP-variant with no signal peptide were compared with control strains without the ASP/GT/GFP-variant encoding genes, respectively, in Example 1 below. The ASP, GT and GFP-variant encoding genes were expressed without a secretion signal which has otherwise been thought to be required for active secretion in Bacillus cells. It was very surprising to find high levels of enzyme activity in the supernatants of the Bacillus hosts. But it was a very welcome surprise that we could produce natively intracellular or non-secreted signal peptide-less polypeptides recombinantly in a Bacillus host cell without having to perform a costly lysis step, since this allows a much more cost-efficient production.

Accordingly, in a first aspect, the present invention provides a method of producing a polypeptide of interest in a Bacillus host cell, said method comprising the steps of:

i) cultivating a Bacillus host cell comprising one or more exogenous polynucleotide encoding a natively non-secreted polypeptide of interest with no secretion signal in a growth medium under conditions conducive to express the polypeptide; and

ii) recovering the polypeptide without performing a lysis step.

In a second aspect, the invention relates to a recombinant Bacillus host cell comprising one or more exogenous or heterologous polynucleotide encoding a natively non-secreted polypeptide of interest with no secretion signal.

### DEFINITIONS

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Cell lysis step: The term “cell lysis step” means a process step introduced during recovery to result in lysis of Bacillus cells. Examples include adding an enzyme, e.g., lysozyme, to the spent fermentation medium; sonication; homogenization, freezing and grinding, and lysis with beads.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Mature polypeptide: The term “mature polypeptide” means a polypeptide in its final form following translation and any post-translational modifications, such as N-terminal processing, C-terminal truncation, etc. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Natively non-secreted polypeptide: The term “natively non-secreted polypeptide” means a polypeptide that is produced intracellularly (i.e., not secreted) by its natural, wild-type source. In the case of a variant, the term “natively non-secreted polypeptide” means that the wild-type polypeptide, from which the variant is derived, is produced intracellularly (i.e., not secreted) by its natural, wild-type source.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

Variant: The term “variant” means a polypeptide having enzyme activity comprising an alteration, i.e., a substitution, insertion, and/or deletion, at one or more (e.g., several) positions compared to the amino acid sequence of its parent wildtype or reference. A substitution means replacement of the amino acid occupying a position with a different amino acid; a deletion means removal of the amino acid occupying a position; and an insertion means adding an amino acid adjacent to and immediately following the amino acid occupying a position.

Variants of a mature polypeptide may comprise a substitution, deletion, and/or insertion at one or more (e.g., several) positions. The number of amino acid substitutions, deletions and/or insertions introduced into the mature polypeptide may be up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for enzyme activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Fusion polypeptides: The polypeptide of interest may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

### DETAILED DESCRIPTION OF THE INVENTION

### Methods of Production

In a first aspect, the present invention relates to methods of producing a natively non-secreted polypeptide of interest in a Bacillus host cell, said method comprising the steps of:

i) cultivating a Bacillus host cell comprising one or more exogenous or heterologous polynucleotides encoding a natively non-secreted polypeptide of interest with no secretion signal in a growth medium under conditions conducive to express the polypeptide; and

ii) recovering the polypeptide without performing a cell-lysis step.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). The temperature is typically in the range of 30-40° C. and the pH is typically in the range of 5-8. The methods of the present invention achieve higher yields of the polypeptide of interest, i.e., at least 1 g/l of fermentation broth, preferably at least 10 g/l and more preferably at least 20 g/l.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Natively Non-Secreted Polypeptides

A natively non-secreted and signal peptide-less polypeptide of interest of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted.

The polypeptide may be a bacterial polypeptide. For example, the polypeptide may be a Gram-positive bacterial polypeptide such as a Bacillus, Clostridium, Enterococcus, Geobacillus, Lactobacillus, Lactococcus, Oceanobacillus, Staphylococcus, Streptococcus, or Streptomyces polypeptide having enzyme activity, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasma polypeptide.

In one aspect, the polypeptide is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis polypeptide.

In another aspect, the polypeptide is a Streptococcus equisimilis, Streptococcus pyogenes, Streptococcus uberis, or Streptococcus equi subsp. Zooepidemicus polypeptide.

In another aspect, the polypeptide is a Streptomyces achromogenes, Streptomyces avermitilis, Streptomyces coelicolor, Streptomyces griseus, or Streptomyces lividans polypeptide.

In another aspect, the polypeptide is derived from a thermophilic or hypothermophilic bacterium from the genera of Aeropylum, Aquifex, Archaeoglubus, Dictyoglomus, Geothermobacterium, Methanopyrus, Pyrococcus, Pyrolobus, Sulpholobus, Thermotoga, and Thermus. A thermophilic organism has an optimum temperature of at least 50° C. and can survive at temperatures up to 70-80° C. A hypothermophilic organism has an optimum temperature of at least 80° C. and requires temperatures of 80-105° C. for growth.

The polypeptide may be a fungal polypeptide. For example, the polypeptide may be a yeast polypeptide such as a Candida, Kluyveromyces, Pichia, Saccharomyces, Schizosaccharomyces, or Yarrowia polypeptide; or a filamentous fungal polypeptide such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria polypeptide.

In another aspect, the polypeptide is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptide.

In another aspect, the polypeptide is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride polypeptide.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.). Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected, the polynucleotide can be isolated or cloned by utilizing techniques that are known to those of ordinary skill in the art (see, e.g., Sambrook et al., 1989, supra).

In a preferred embodiment of the invention, the polypeptide of interest is an enzyme; preferably the enzyme is a hydrolase, isomerase, ligase, lyase, oxidoreductase, or transferase; more preferably the enzyme is an alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, green fluorescent protein, glucanotransferase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or a xylanase.

In a preferred embodiment the enzyme is resistant to degradation by one or more endogenous proteases produced by the Bacillus host cell in the fermentation broth. Resistance to degradation is defined as an accumulation of the enzyme in the supernatant over time during fermentation in the defined media described in Example 1.

In a preferred embodiment the enzymes are derived from a thermophilic or hypothermophilic organism. Enzymes from these organisms are generally more stable at high temperatures (50-110° C.) and also show a higher resistance towards proteases at lower temperatures because of their constrained structures (Parcell and Sauer, 1989; Daniel et. al, 1982 Pacell and Sauer, 1989, J. Biol. Chem. 264: 7590-7595; Danel et al., 1982, Biochem. J. 207: 641-644).

In a more preferred embodiment of the invention the polypeptide of interest is an enzyme; more preferably it is a glucanotransferase comprising or consisting of an amino acid sequence at least 70% identical to SEQ ID NO:1, preferably at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO:1, a variant of the green fluorescent protein from the marine jellyfish Aequorea victoria comprising or consisting of an amino acid sequence at least 70% identical to SEQ ID NO:2, preferably at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO:2, or an asparaginase comprising or consisting of an amino acid sequence at least 70% identical to SEQ ID NO:3, preferably at least 75%, at least 80%, at least 85%, at least 90%, at least 91%, at least 92%, at least 93%, at least 94%, at least 95%, at least 96%, at least 97%, at least 98%, at least 99%, or 100% identical to SEQ ID NO:3.

### Polynucleotides

The present invention also relates to exogenous or heterologous polynucleotides encoding a natively non-secreted polypeptide of interest with no signal peptide has described herein.

The techniques used to isolate or clone a polynucleotide are known in the art and include isolation from genomic DNA or cDNA, or a combination thereof. The cloning of the polynucleotides from genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of a polynucleotide encoding a polypeptide comprising or consisting of the amino acid sequence shown in SEQ ID NO: 1, SEQ ID NO: 2 or SEQ ID NO: 3, a subsequence thereof, and/or by introduction of nucleotide substitutions that do not result in a change in the amino acid sequence of the polypeptide, but which correspond to the codon usage of the host organism intended for production of the polypeptide, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell. Preferably, each of the one or more exogenous or heterologous polynucleotides of the invention is operably linked with at least one promoter to enable its expression; preferably the promoter is endogenous or exogenous; more preferably the promoter is a tandem promoter. Examples of tandem promoters are disclosed in WO 99/43835.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a propeptide coding sequence that encodes a propeptide positioned, e.g., at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. Other examples of regulatory sequences are those that allow for gene amplification.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated; preferably the one or more exogenous polynucleotide of the invention is integrated into at least one chromosomal locus of the host cell; preferably into several loci. A polynucleotide or a vector may be chromosomally integrated in two or more copies as shown in WO 2006/042548. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant Bacillus host cells comprising one or more exogenous or heterologous polynucleotide encoding a natively non-secreted polypeptide of interest with no secretion signal. The polynucleotide of the present invention is operably linked to one or more control sequences that direct the production of the polypeptide of interest. A construct or vector comprising the polynucleotide is introduced into a host cell so that the construct or vector is maintained as a chromosomal integrant or as a self-replicating extrachromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). However, any method known in the art for introducing DNA into a host cell can be used.

### Fermentation Broth Formulations or Cell Compositions

The present invention also relates to a fermentation broth formulation or a cell composition comprising a host cell of the present invention. The fermentation broth product further comprises additional ingredients used in the fermentation process, such as, for example, cells (including, the host cells containing the exogenous or heterologous polynucleotide(s) encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### EXAMPLES

### Example 1

### Production of Glucanotransferase (GT), Green Fluorescent Protein (GFP) and Asparaginase (ASP) without Signal Peptide

### Strains:

### PP2577-1:

A derivative of a parent Bacillus licheniformis Si3 strain which in turn is a sporulation deficient derivative of a natural isolate of Bacillus licheniformis Ca63. The taxonomic characteristics of the Bacillus licheniformis strain, Ca63, are the following: Name=Bacillus licheniformis, Order=Bacillaceae, Genus=Bacillus, Species=licheniformis. (Ref.: Bergey's Manual of Systematic Bacteriology). The classification of the host strain is based on the taxonomic characteristics given in Priest and Alexander, 1988, Journal of General Microbiology 134: 3011-3018. The PP2577-1 derivative has disruptive gene deletions achieved via double homologous recombinations into the genes encoding the following gene products: Mpr (metalloprotease), AprE (alkaline protease), BglC (endo-1,4-beta-glucanase), and AmyE (alpha-amylase) (names refer to the corresponding homologues in Bacillus subtilis). Production of the glucanotransferase, GT derived from Thermus thermophilus, is obtained from three integrated expression cassettes on the chromosome (as done in U.S. Pat. No. 6,255,076). The expression cassettes are inserted in or next to the amyE (alpha-amylase), the gntP (gluconate permease) and the xylA (xylose isomerase) loci (as done in EP 1805296).

### PP2913-1:

A sporulation deficient Bacillus licheniformis strain Si3 derivative with disruptive gene deletions achieved via double homologous recombinations into the genes encoding the following gene products: Mpr (metalloprotease), AprE (alkaline protease), Vpr (minor extracellular serine protease), Bpr (bacillopeptidase F), Epr (minor extracellular serine protease), BglC (endo-1,4-beta-glucanase), and AmyE (alpha-amylase) (names refer to the corresponding homologues in Bacillus subtilis). Production of the green fluorescent protein BioST, is obtained from a chromosomal integrated expression cassette inserted next to the glpD (glycerol-3-phosphate-dehydrogenase) loci.

### MOL2940:

A sporulation deficient Bacillus subtilis A164 strain ATCC 6051a with disruptive gene deletions achieved via double homologous recombinations into the genes encoding the following gene products: AmyE (alpha-amylase), BglC (endo-1,4-beta-glucanase), Pel (pectate lyase), AprE (alkaline protease), NprE (extracellular neutral protease B), Bpr (bacillopeptidase F), Vpr (minor extracellular serine protease), Mpr (metallprotease), Epr (minor extracellular serine protease) and WprA (secreted quality control protease) (names refer to the corresponding homologues in Bacillus subtilis). Production of the asparaginase AsnPfu from Pyrococcus furiosus ATCC 43587, is obtained from three integrated expression cassettes on the chromosome. The expression cassettes are inserted in or next to the alr (alanine racemase), pel (pectate lyase), and amyE (alpha-amylase) loci.

A number of fed-batch fermentation process with Bacillus licheniformis and Bacillus subtilis were conducted as described below. All media were sterilized by methods known in the art. Unless otherwise described, tap water was used. The ingredient concentrations referred to in the below recipes are before any inoculation.

### First Inoculum Medium:

LB agar: 10 g/l peptone from casein; 5 g/l yeast extract; 10 g/l sodium chloride; 12 g/l Bacto-agar adjusted to pH 6.8 to 7.2. Premix from Merck was used.

### Transfer Buffer:

M-9 buffer (deionized water is used for the preparation): di-sodium hydrogen phosphate, 2H₂O 8.8 g/l; potassium dihydrogen phosphate 3 g/l; sodium chloride 4 g/l; magnesium sulphate, 7H₂O 0.2 g/l.

### Inoculum Shake Flask Medium (Concentration is Before Inoculation):

PRK-50: 110 g/l soy grits; Di-Sodiumhydrogenphosphate, 2H₂O 5 g/l; pH adjusted to 8.0 with NaOH/H₃PO₄ before sterilization.

### Make-Up Medium (Concentration is Before Inoculation):

Tryptone (Casein hydrolysate from Difco) 30 g/l; Magnesium sulphate, 7H₂O 4 g/l; di-potassium hydrogen phosphate 7 g/l; di-sodium hydrogen phosphate, 2H₂O 7 g/l; di-ammonium sulphate 4 g/l; citric acid 0.78 g/l; vitamins (thiamin-dichloride 34.2 mg/l; riboflavin 2.9 mg/l; nicotinic acid 23 mg/l; calcium D-pantothenate 28.5 mg/l; Pyridoxal-HCl 5.7 mg/l; D-biotin 1.1 mg/l; folic acid 2.9 mg/l); trace metals (MnSO₄, H₂O 39.2 mg/l; FeSO₄, 7H₂O 157 mg/l; CuSO₄, 5H₂O 15.6 mg/l; ZnCl₂ 15.6 mg/l); antifoam (SB2121) 1.25 ml/l; pH adjusted to 6.0 with NaOH/H₃PO₄ before sterilization.

### Feed Medium:

### Sucrose 708 g/l

### Procedure for Inoculum Steps:

First the strain was grown on LB agar slants 1 day at 37° C.

The agar was then washed with M-9 buffer, and the optical density (OD) at 650 nm of the resulting cell suspension was measured.

The inoculum shake flask (PRK-50) was inoculated with an inoculum of OD (650 nm)×ml cell suspension=0.1.

The shake flask was incubated at 37° C. at 300 rpm for 20 hours.

The fermentation in the main fermentor (fermentation tank) was started by inoculating the main fermentor with the growing culture from the shake flask. The inoculated volume was 11% of the make-up medium (80 ml for 720 ml make-up media).

### Fermentor Equipment:

Standard lab fermentors were used equipped with a temperature control system, pH control with ammonia water and phosphoric acid, dissolved oxygen electrode to measure >20% oxygen saturation through the entire fermentation.

### Fermentation Parameters:

### Temperatures: 41° C. (GFP, GT); 43° C. (ASP).

The pH was kept between 6.8 and 7.2 using ammonia water and phosphoric acid
Control: 6.8 (ammonia water); 7.2 phosphoric acid
Aeration: 1.5 liter/min for a starting volume of 800 ml after inculation.

### Agitation: 1500 rpm

### Feed Strategy:

0 hour. 0.05 g/min/kg initial broth after inoculation
8 hours. 0.156 g/min/kg initial broth after inoculation
End 0.156 g/min/kg initial broth after inoculation

### Asparaginase Assay:

The activity of asparaginase may be measured in ASNU. One asparaginase unit (ASNU) is defined as the amount of enzyme needed to generate 1.0 micromole of ammonia in 1 minute at 37° C. and pH 7.0, in 0.1 M MOPS buffer with 9.2 mg/ml asparagine.

Asparaginase hydrolyzes asparagine to aspartic acid and ammonia. The asparaginase activity is determined in a coupled assay where the ammonia produced is reacted with α-ketoglutarate and NADH to form glutamic acid and NAD⁺. The latter reaction is catalyzed by glutamate dehydrogenase, which is added in surplus. One mole of ammonia produced will result in consumption of one mole of NADH. The consumption of NADH is measured spectrofotometrically at 340 nm. NADH has an absorbance at 340 nm (molar extension coefficient 6300 (M*cm)⁻¹), while NAD+ has no significant absorbance at this wavelength. A decrease in color is thus measured, and can be correlated to asparaginase activity.

### Assay Conditions:

| | |
|---|---|
| Temperature: | 37.0 ± 0.5° C. |
| pH: | 7.00 ± 0.05 (at room temperature) |
| L-asparagine: | 9.2 mg/ml |
| Enzyme working range: | 0.0207-0.0775 ASNU/ml |
| Interval kinetic measuring time: | 2 minutes |
| | Incubate 1.5 minutes before measuring |
| Wavelength: | 340 nm |
| α-ketoglutarate: | 2.3 mg/ml |
| NADH: | 0.405 mg/ml |
| GIDH glutamate dehydrogenase | Minimum 61.8 U/ml |
| (from bovine liver) (EC 1.4.1.3): | |

### Assay Procedure:

1) Dilution of samples: Weigh out approx. 1 g of the sample. Dissolve and make up to the mark in a 100 mL measuring flask with buffer containing 0.1 M sodium acetate and 0.225 g/L Brij 35, pH 5.0. Stir the solution on magnetic stirrer for 15-30 minutes before diluting further to approx. 0.57 ASNU/mL.

2) Preparation of substrate-reagent solution: Make a solution containing 66.6 mM L-asparagine, 0.66 mM NADH, 13.3 mM alpha-ketoglutarate and 67.2 U/mL glutamate dehydrogenase in 0.1 M MOPS, pH 7.0. The solution should be protected from light e.g. by wrapping the flask containing the substrate reagent in foil. This reagent is stable for about 6 hours at room temperature, if the initial absorbance is above 2.3 absorbance units at 340 nm.

3) The measurement is started by pipetting 230 μL substrate-reagent solution into a UV-transparent microtiter plate and pre-incubate the plate for 8 minutes at 37° C. after which 20 μL of the sample is added and the mixture is further equilibrated for 90 seconds at 37° C. The absorbance is then read at 340 nm for 120 seconds, e.g. every 18 seconds.

4) The activity is determined from the slope of a plot of the absorbance versus time and is related to a standard curve.

### Chemicals:

| Name | Formula | Brand |
|---|---|---|
| MOPS: | C₇H₁₅NO₄S | E.g., Sigma M-1254 |
| (abbreviation for 3- | | |
| morpholinopropanesulfonic acid): | | |
| Brij 35, 30% w/v | CH₃(CH₂)₁₀CH₂(OCH₂CH₂)ₙOH | E.g., Sigma B4184 |
| (polyoxyethyleneglycol-dodecyl | | |
| ether): | | |
| 4M sodium hydroxide: | NaOH | E.g., Fluka 35274 |
| L-asparagine: | C₄H₈N₂O₃ | E.g., Sigma A-7094 |
| NADH (Nicotinamide adenine | C₂₁H₂₇N₇O₁₄P₂ | E.g., Roche 107735 |
| dinucleotide): | | |
| α-ketoglutarate: | C₅H₄O₅Na₂ | E.g. Sigma K-3752 |
| Glutamate dehydrogenase | | E.g., Sigma 2626 |
| (GIDH from bovine liver): | | |
| Sodium acetate: | C₂H₃NaO₂*3H₂0 | E.g., Sigma Aldrich |
| | | 32318 |
| Acetic acid, concentrated: | C₂H₄O₂ | E.g., Fluka 33209 |
| Ultrapure water: | H₂O with resistivity ≧18.2 MΩ * | E.g., Millipore |
| | cm at 25° C. | |

### Sample Preparation:

1. Weigh out approx. 1 g of the sample.
2. Dissolve and fill up to the mark in a 100-ml measuring flask with 0.1 M acetate buffer pH 5.0 with Brij.
3. Stir on a magnetic stirrer for 15-30 minutes.
4. Dilute the samples further with 0.1 M acetate buffer pH 5.0 with Brij to approx. 0.57 ASN U/ml.

Calculation is based on the molar extension on NADH, and the fact that there is generated equimolary amount of NAD+ compared to the amount of asparagine acid converted.

The enzyme activity is then in this case correlated relative to the end sample.

When measuring total enzyme activity in samples no separation of the sample is done prior to the dilution and enzymatic analysis. In the case of measuring of enzyme activity in the supernatant 10 ml culture broth samples are centrifuged at 8500 g for 30 minutes, the supernatant is decanted from the precipitate, and sterile filtered through a 0.22 or 0.45 μm filter, to ensure it does not contain any cells, before the dilution and determination of the enzyme activity. If the enzyme is mostly present in the supernatant, higher activity will be determined in this fraction compared to the total sample, as the biomass in that case will dilute the enzyme activity.

### Glucanotransferase Assay

One GTNU (Glucano Transferase Unit) is the amount of enzyme that produces 1 μmol of Glucose per minute under the above described conditions, but at 60° C. This corresponds to the amount of enzyme that produces 1 μmol glucose per minute at 37° C. multiplied by a factor around 7.7.

Glucanotransferase (EC 2.4.1.25) is an enzyme that transfers a 1,4-α-D-Glucan to a new 4 position, that must be glucose or another 1,4-α-D-Glucan. Maltotriose is used as substrate in this assay and the activity of Glucanotransferase is measured as production of glucose in the reaction below:

G-G-G+G-G-G->G-G-G-G-G+G

The glucanotransferase reaction is stopped and the enzyme inactivated by addition of Sodium hydroxide.

The formation of glucose is measured using a commercial kit from Thermo Electron Corporation. Glucose is phosphorylated by hexokinase into glucose-6-phosphate and further oxidised by glucose-6-phosphate dehydrogenase to 6-phosphogluconate whereby NAD+ is reduced to NADH. The formation of NADH is measured at 340 nm. Alternative ways of measuring the generated glucose can also be used.

### Assay Conditions:

| | |
|---|---|
| Phosphate buffer: | 50 mM |
| pH: | 6.0 |
| Maltotriose: | 0.86% W/V |
| Dextrin: | 0.05% W/V |
| Temperature: | 37° C. |
| Reaction time: | 5 minutes |
| Wavelength: | 340 nm |
| Enzyme working range: | 0.558-1.67 GTNU/mL |

### Assay Procedure:

1) Dilution of samples: Weigh out approx. 1 g of the sample. Dissolve and make up to the mark in a 100 mL measuring flask with buffer containing 50 mM Phosphate buffer and 0.1% W/V Triton X-100, pH 6.0. Stir the solution on magnetic stirrer for 15-30 minutes.

2) Incubate the diluted sample 15 minutes at 62° C.+1-2° C. in order to inactivate any α-glucosidases.

3) The sample is further diluted to a enzyme activity of approx. 1.1 GTNU/mL.

4) Preparation of substrate-reagent solution: Make a solution containing 1% W/V maltodextrin and 0.06% W/V dextrin in a buffer containing 50 mM phosphate buffer and 0.1% W/V Triton X-100, pH 6.0

5) The measurement is started by pipetting 77 μL substrate-reagent solution into a UV-transparent microtiter plate and pre-incubate the plate 8 minutes at 37° C. after which 13 μL of the sample is added and the mixture is incubated for 5 minutes at 37° C. The reaction is stopped by adding 13 μL 0.5 M NaOH. The amount of glucose produced is measured e.g. by adding 147 μL reaction mix from the commercial kit from Thermo Electron Corporation. The reaction is allowed to run for 280 seconds and the absorbance is then read at 340 nm.

6) The activity is determined from the measured absorbance related to a standard curve.

### Chemicals:

| Name | Formula | Brand |
|---|---|---|
| Potassium | KH₂PO₄ | e.g. Merck 4873 |
| dihydrogen | | |
| phosphate: | | |
| Triton X-100: | t-Oct-C₆H₄—(OCH₂)ₓOH x = 9-10 | e.g. Sigma T-9284 |
| Maltotriose: | C₁₈H₃₂O₁₆ | e.g. Sigma M-8378 |
| Sodium | NaOH | e.g. Merck 6498 |
| hydroxide: | | |
| Glucose | — | Thermo Fisher |
| determination: | | Scientific 981304 |
| | | or 981779 |
| Dextrin: | (C₆H₁₂O₆)x | Avedex W80 |
| | | ATD chemical |
| | | no. 040 |

### Sample Preparation:

1. Weigh out approx. 1 g of the sample.
2. Dissolve and fill up to the mark in a 100-ml measuring flask with buffer containing 50 mM Phosphate buffer and 0.1% W/V Triton X-100, pH 6.0.
3. Stir on a magnetic stirrer for 15-30 minutes.
4. Incubate the diluted sample 15 minutes at 62° C.+/−2° C. in order to inactivate any α-glucosidases.
5. The samples are further diluted with buffer containing 50 mM phosphate buffer and 0.1% W/V Triton X-100, pH 6.0.

The sample is first incubated 300 seconds with the maltotriose, the reaction is then stopped by adding NaOH, and the amount of glucose is then detected by the commercial kit from Thermo Electron Corporation. Reaction time for the second reaction is 280 seconds, and then absorbance is measured at 340 nm. The enzyme activity is then in this case correlated relative to the end sample.

When measuring total enzyme activity in samples no separation of the sample is done prior to the dilution and enzymatic analysis. In the case of measuring of enzyme activity in the supernatant samples are centrifuged at 8500 g for 30 minutes, the supernatant is decanted from the precipitate, and sterile filtered through a 0.22 μm filter before the dilution and determination of the enzyme activity. If the enzyme is mostly present in the supernatant, higher activity will be determined in this fraction compared to the total sample, as the cell in that case will dilute the enzyme activity.

### Results:

The fermentation was run for three to five days and the results are shown in Tables 1-3.

TABLE 1
Glucanotransferase 2*⁾

| Time | Total in broth | In supernatant |
|---|---|---|
| day 1 | 11 | 3 |
| day 2 | 35 | 22 |
| day 3 | 54 | 45 |
| day 4 | 84 | 84 |
| day 5 | 100 | 104 |

*⁾Amount of activity (GTNU) was determined as shown elsewhere herein and normalized to 100% for total activity in the broth at day 5.

TABLE 2
GFP*⁾

| Time | Total in broth | In supernatant |
|---|---|---|
| day 1 | 61 | 8 |
| day 2 | 119 | 34 |
| day 3 | 100 | 84 |

*⁾The amount of protein was determined using fluorescence spectroscopy measurement for determination of GFP. GFP is well-known to many working with protein expression and it is known that it can be directly measured even when present in the cells. The amount determined by fluorescence spectroscopy of the total broth therefore corresponds to the protein content of both the cells and the supernatant. The amount was normalized to 100% for the total activity in the broth at day 3.

TABLE 3
Asparaginase*⁾

| Time | Total in broth | In supernatant |
|---|---|---|
| day 1 | 26 | 0 |
| day 2 | 54 | 0 |
| day 3 | 68 | 40 |
| day 4 | 101 | 83 |
| day 5 | 100 | 81 |

*⁾Amount of activity (ASNU) was determined as shown elsewhere herein and normalized to 100% for total activity in the broth at day 5.

Contrary to our expectations, we found very high levels of enzyme activity in the supernatants when natively intracellular or non-secreted enzymes were expressed in Bacillus licheniformis and Bacillus subtilis. This is a highly surprising result and one of economical importance, since this means natively non-secreted polypeptides can now be produced in Bacillus and be recovered directly from the broth without an otherwise needed and costly cell lysis step.

The invention is further defined in the following paragraphs:

1. A method of producing a natively non-secreted polypeptide in a Bacillus host cell, said method comprising the steps of:

(a) cultivating a Bacillus host cell comprising one or more exogenous or heterologous polynucleotides encoding the natively non-secreted polypeptide with no secretion signal in a growth medium under conditions conducive to express the natively non-secreted polypeptide; and

(b) recovering the natively non-secreted polypeptide without performing a cell-lysis step.

2. A method of producing a polypeptide, comprising the steps of:

(a) cultivating a Bacillus host cell comprising one or more polynucleotides encoding the polypeptide under conditions conducive for production of the polypeptide, wherein the polypeptide is produced intracellulary by its native, wild-type source and wherein the one or more polynucleotides are not operably linked to a signal peptide coding sequence; and

(b) recovering the polypeptide without performing a cell-lysis step.

3. The method of paragraph 1 or 2, wherein the Bacillus host cell is selected from the group consisting of Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis.
4. The method of any of paragraphs 1-3, wherein the polypeptide is native to the Bacillus host cell.
5. The method of any of paragraphs 1-3, wherein the polypeptide is heterologous to the Bacillus host cell.
6. The method of any of paragraphs 1-3 and 5, wherein the polypeptide is derived from a thermophilic organism.
7. The method of any of paragraphs 1-3 and 5, wherein the polypeptide is derived from a hypothermophilic organism.
8. The method of paragraph 6 or 7, wherein the thermophilic or hypothermophilic organism is selected from the group consisting of Aeropylum, Aquifex, Archaeoglubus, Dictyoglomus, Geothermobacterium, Methanopyrus, Pyrococcus, Pyrolobus, Sulpholobus, Thermotoga, and Thermus.
9. The method of any of paragraphs 1-8, wherein the polypeptide is resistant to degradation by one or more endogenous proteases produced by the Bacillus host cell in the fermentation broth.
10. The method of any of paragraphs 1-9, wherein the polypeptide of interest is an enzyme; preferably the enzyme is a hydrolase, isomerase, ligase, lyase, oxidoreductase, or transferase; more preferably the enzyme is an alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, green fluorescent protein, glucanotransferase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or a xylanase.
11. The method of paragraph 10, wherein the enzyme is a glucanotransferase comprising an amino acid sequence that is at least 70% identical to SEQ ID NO:1 or a variant of the green fluorescent protein from the marine jellyfish Aequorea victoria comprising an amino acid sequence that is at least 70% identical to SEQ ID NO:2 or an asparaginase comprising an amino acid sequence that is at least 70% identical to SEQ ID NO:3.
12. The method of any of paragraphs 1-11, wherein each of said one or more polynucleotides is operably linked with at least one promoter to enable its expression; preferably the promoter is endogenous or exogenous; more preferably the promoter is a tandem promoter.
13. The method of any of paragraphs 1-12, wherein the one or more polynucleotides are integrated into at least one chromosomal locus of the Bacillus host cell; preferably into several loci.
14. The method of any of paragraphs 1-13, wherein the polypeptide is recovered by collection, centrifugation, filtration, extraction, spray-drying, evaporation, or precipitation.
15. The method of any of paragraphs 1-13, wherein the polypeptide is recovered in the fermentation broth.
16. The method of any of paragraphs 1-15, wherein the Bacillus host cell comprises a disruption, e.g., a deletion, of one or more nucleic acid sequences encoding one or more proteases, e.g., four, five or six proteases, that results in the production of less protease.
17. The method of paragraph 16, wherein the one or more proteases are selected from the group consisting of AprE (alkaline protease), Bpr (bacillopeptidase F), Epr (minor extracellular serine protease), Mpr (metalloprotease), NprE (extracellular neutral protease B), Vpr (minor extracellular serine protease), and WprA (secreted quality control protease).
18. The method of any of claims 1-17, wherein the amount of polypeptide produced is at least 1 g/l of fermentation broth, preferably at least 10 g/l and more preferably at least 20 g/l.
19. A recombinant Bacillus host cell comprising one or more exogenous or heterologous polynucleotide encoding a natively non-secreted polypeptide of interest with no secretion signal.
20. A recombinant Bacillus host cell comprising one or more polynucleotides encoding a polypeptide, wherein the polypeptide is produced intracellulary by its native, wild-type source and wherein the one or more polynucleotides are not operably linked to a signal peptide coding sequence.
21. The host cell of paragraph 19 or 20, wherein the Bacillus host cell is selected from the group consisting of Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis.
22. The host cell of any of paragraphs 19-21, wherein the polypeptide is native to the Bacillus host cell.
23. The host cell of any of paragraphs 19-21, wherein the polypeptide is heterologous to the Bacillus host cell.
24. The host cell of any of paragraphs 19-21 and 23, wherein the polypeptide is derived from a termophilic organism.
25. The host cell of any of paragraphs 19-21 and 23, wherein the polypeptide is derived from a hypothermophilic organism.
26. The host of paragraph 24 or 25, wherein the thermophilic or hypothermophilic organism is selected from the group consisting of Aeropylum, Aquifex, Archaeoglubus, Dictyoglomus, Geothermobacterium, Methanopyrus, Pyrococcus, Pyrolobus, Sulpholobus, Thermotoga, and Thermus.
27. The host cell of any of paragraphs 19-26, wherein the polypeptide is resistant to degradation by one or more endogenous proteases produced by the Bacillus host cell in the fermentation broth.
28. The host cell of any of paragraphs 19-27, wherein the polypeptide of interest is an enzyme; preferably the enzyme is a hydrolase, isomerase, ligase, lyase, oxidoreductase, or transferase; more preferably the enzyme is an alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, green fluorescent protein, glucanotransferase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or a xylanase.
29. The host cell of paragraph 28, wherein the enzyme is a glucanotransferase comprising an amino acid sequence that is at least 70% identical to SEQ ID NO:1 or a variant of the green fluorescent protein from the marine jellyfish Aequorea victoria comprising an amino acid sequence that is at least 70% identical to SEQ ID NO:2 or an asparaginase comprising an amino acid sequence that is at least 70% identical to SEQ ID NO:3.
30. The host cell of any of paragraphs 19-29, wherein each of the one or more polynucleotides is operably linked with at least one promoter to enable its expression; preferably the promoter is endogenous or exogenous; more preferably the promoter is a tandem promoter.
31. The host cell of any of paragraphs 19-30, wherein the one or more polynucleotides are integrated into at least one chromosomal locus of the Bacillus host cell; preferably into several loci.
32. The host cell of any of paragraphs 19-31, which further comprises a disruption, e.g., a deletion, of one or more nucleic acid sequences encoding one or more proteases, e.g., four, five or six proteases, that results in the production of less protease.
33. The host cell of paragraph 32, wherein the one or more proteases are selected from the group consisting of AprE (alkaline protease), Bpr (bacillopeptidase F), Epr (minor extracellular serine protease), Mpr (metalloprotease), NprE (extracellular neutral protease B), Vpr (minor extracellular serine protease), and WprA (secreted quality control protease).

1. A method of producing a natively non-secreted polypeptide in a Bacillus host cell, said method comprising the steps of:
(a) cultivating a Bacillus host cell comprising one or more exogenous or heterologous polynucleotides encoding the natively non-secreted polypeptide with no secretion signal in a growth medium under conditions conducive to express the natively non-secreted polypeptide; and
(b) recovering the natively non-secreted polypeptide without performing a cell-lysis step.

2. A method of producing a polypeptide, comprising the steps of:
(a) cultivating a Bacillus host cell comprising one or more polynucleotides encoding the polypeptide under conditions conducive for production of the polypeptide, wherein the polypeptide is produced intracellulary by its native, wild-type source and wherein the one or more polynucleotides are not operably linked to a signal peptide coding sequence; and
(b) recovering the polypeptide without performing a cell-lysis step.

3. The method of claim 1 or 2, wherein the Bacillus host cell is selected from the group consisting of Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis, preferably Bacillus licheniformis or Bacillus subtilis.

4. The method of claim 1, wherein the polypeptide is native to the Bacillus host cell.

5. The method of claim 1, wherein the polypeptide is heterologous to the Bacillus host cell.

6. The method of claim 1, wherein the polypeptide is derived from a thermophilic organism.

7. The method of claim 1, wherein the polypeptide is derived from a hypothermophilic organism.

8. The method of claim 6, wherein the thermophilic or hypothermophilic organism is selected from the group consisting of Aeropylum, Aquifex, Archaeoglubus, Dictyoglomus, Geothermobacterium, Methanopyrus, Pyrococcus, Pyrolobus, Sulpholobus, Thermotoga, and Thermus.

9. The method of claim 1, wherein the polypeptide is resistant to degradation by one or more endogenous proteases produced by the Bacillus host cell in the fermentation broth.

10. The method of claim 1, wherein the polypeptide is an enzyme; preferably the enzyme is a hydrolase, isomerase, ligase, lyase, oxidoreductase, or transferase.

11. The method of claim 10, wherein the enzyme is a glucanotransferase comprising an amino acid sequence that is at least 70% identical to SEQ ID NO:1 or a variant of the green fluorescent protein from the marine jellyfish Aequorea victoria comprising an amino acid sequence that is at least 70% identical to SEQ ID NO:2 or an asparaginase comprising an amino acid sequence that is at least 70% identical to SEQ ID NO:3.

12. The method of claim 1, wherein each of said one or more polynucleotides are operably linked with at least one promoter to enable its expression.

13. The method of claim 1, wherein the one or more polynucleotides are integrated into at least one chromosomal locus of the Bacillus host cell; preferably into several loci.

14. The method of claim 1, wherein the polypeptide is recovered by collection, centrifugation, filtration, extraction, spray-drying, evaporation, or precipitation.

15. The method of claim 1, wherein the polypeptide is recovered in the fermentation broth.

16. The method of claim 1, wherein the Bacillus host cell comprises a disruption of one or more nucleic acid sequences encoding one or more proteases that results in the production of less protease.

17. The method of claim 16, wherein the one or more proteases are selected from the group consisting of AprE (alkaline protease), Bpr (bacillopeptidase F), Epr (minor extracellular serine protease), Mpr (metalloprotease), NprE (extracellular neutral protease B), Vpr (minor extracellular serine protease), and WprA (secreted quality control protease).

18. The method of claim 1, wherein the amount of polypeptide produced is at least 1 g/l of fermentation broth.

19. A recombinant Bacillus host cell comprising one or more exogenous or heterologous polynucleotide encoding a natively non-secreted polypeptide with no secretion signal.

20. (canceled)
